(19) United States
(12) Patent Application Publication
(10) Pub. No.: US 2022/0053761 A1
(43) Pub. Date: Feb. 24, 2022

(54) BIOLOGICAL-BASED CATALYST TO DELAY PLANT DEVELOPMENT PROCESSES

(71) Applicant: CROWPIERCE TECHNOLOGIES, LLC, Canton, GA (US)

(72) Inventors: Sidney Crow, JR., Smyrna, GA (US); George Pierce, Canton, GA (US)

(21) Appl. No.: 17/406,761

(22) Filed: Aug. 19, 2021

Related U.S. Application Data
(60) Provisional application No. 63/067,866, filed on Aug. 19, 2020.

(51) Int. Cl. A01N 37/46; C12N 9/02; C12N 9/14; A01N 63/20

(52) U.S. Cl. CPC A01N 37/46; C12N 9/0073; C12N 9/14; C12Y 114/14011; C12Y 303/02003; C12Y 114/13069; A01N 63/20

(57) ABSTRACT

Disclosed are purified proteins, cell lysates, cell-free extracts, and protein extracts comprising 1-hexene monooxygenase (1-HMO) and methods of their use for delaying plant development.

This application claims the benefit of U.S. Provisional Application No. 63/067,866, filed on Aug. 19, 2020, which is incorporated herein by reference in its entirety.

### I. BACKGROUND

Ethylene production (biosynthesis) in plants and plant parts is induced by a variety of external factors and stressors, including wounding, the application of hormones (e.g., auxin), anaerobic conditions, chilling, heat, drought, and pathogen infection. Increased ethylene production also is observed during a variety of plant development processes, including fruit or vegetable ripening, seed germination, leaf abscission, and flower senescence.

Ethylene biosynthesis in plants is an end product that is typically depicted as an enzymatic scheme, which is accomplished during the pathway traditionally referred to as the “Yang Cycle,” in which S-adenosyl-L-methionine (SAM) synthase catalyzes conversion of methionine to S-adenosyl-L-methionine (AdoMet); 1-aminocyclopropane-1-carboxylic acid (ACC) synthase catalyzes the conversion of AdoMet to ACC; and ACC oxidase catalyzes the conversion of ACC to ethylene and the byproducts carbon dioxide and hydrogen cyanide. See, for example, Srivastava (2001) Plant Growth and Development: Hormones and Environment (Academic Press, New York) for a general description of ethylene biosynthesis in plants and plant development processes regulated by ethylene.

Previous research has established that in climacteric fruit, ripening is triggered, at least in part, by a sudden and significant increase in ethylene biosynthesis and respiration. Although a sudden burst of ethylene production is strongly implicated in the fruit ripening process of climacteric fruits, the exact mechanism, particularly in nonclimacteric fruits, is not completely understood. While there is no sudden burst of ethylene production in non-climacteric fruit, many non-climacteric fruit will respond to ethylene (and some very much more than others). Moreover, fruits, vegetables, and other plant products vary in the amount of ethylene synthesized and also in the sensitivity of the particular product to ethylene. For example, apples exhibit a high level of ethylene production and ethylene sensitivity, whereas artichokes display a low level of ethylene biosynthesis and ethylene sensitivity. See, for example, Cantwell (2001) “Properties and Recommended Conditions for Storage of Fresh Fruits and Vegetables” at postharvest.ucdavis.edu/Produce/Storage/index.shtml (last accessed on Mar. 6, 2007), which is herein incorporated by reference in its entirety. Fruit ripening typically results in a change in color, softening of the pericarp, and changes in the sugar content and flavor of the fruit. While ripening initially makes fruit more edible and often more attractive to eat, the process eventually leads to degradation and deterioration of fruit quality, making it unacceptable for consumption and susceptible to microbial deterioration, especially by fungi, leading to significant commercial monetary losses. Susceptibility to fungal attack occurs early in the process and is exacerbated by physical trauma, including the physical trauma that can occur during handling and shipping. Control of the ripening process is desirable for improving shelf-life and extending the time available for transportation, storage, and sale of fruit and other agricultural products subject to ripening.

In addition to a sudden increase in ethylene biosynthesis in climacteric fruits, ripening-related changes are also associated with a rise in respiration rate and susceptiblity to microbial deterioration, especially by fungi. Heat is produced as a consequence of respiration in fruit, vegetables, and other plant products and, consequently, impacts the shelf-life and the required storage conditions (e.g., refrigeration) for these commodities. Plant products with higher rates of respiration (e.g., artichokes, cut flowers, asparagus, broccoli, spinach, etc.) exhibit shorter shelf-lives than those with lower respiration rates (e.g., nuts, dates, apples, citrus fruits, grapes, etc.). Respiration is affected by a number of environmental factors including temperature, atmospheric composition, physical stress, light, chemical stress, radiation, water stress, growth regulators, and pathogen attack. In particular, temperature plays a significant role in respiration rate. For a general description of respiratory metabolism and recommended controlled atmospheric conditions for fruits, vegetables, and other plant products.

Methods and compositions for delaying the fruit ripening process include, for example, the application of silver salts (e.g., silver thiosulfate), 2,5-norbornadiene, potassium permanganate, 1-methylcyclopropene (1-MCP), cyclopropene (CP) and derivatives thereof. These compounds have significant disadvantages, such as the presence of heavy metals, foul odors, and explosive properties when compressed, that make them unacceptable for or of limited applicability for use in the food industry. EdibleWax coating (Decco 1930s) have been used for almost 100 years to mitigate deterioration effect of ripening by retarding water loss which occurs during respiration. Transgenic approaches for controlling ethylene production to delay plant development processes (e.g., fruit ripening) by introducing nucleic acid sequences that limit ethylene production, particularly by reducing the expression of the enzymes ACC synthase or ACC oxidase, are also under investigation. The public's response to genetically modified agricultural products, however, has not been entirely favorable.

Accordingly, a significant need remains in the art for safe methods and apparatuses to delay plant development processes. Such methods and apparatuses could provide better control of fruit ripening, vegetable ripening, flower senescence, leaf abscission, and seed germination and extend the shelf-life of various agricultural products (e.g., fruit, vegetables, and cut flowers), thereby permitting longer distance transportation of these products without the need for refrigeration, increasing product desirability to consumers, and decreasing monetary costs associated with product loss due to untimely ripening and senescence.

### II. SUMMARY

Disclosed herein are cell lysate and cell-free extracts of Rhodococcus rhodochrous DAP 96253 and methods of their use.

In one aspect, disclosed herein are cell lysates, cell-free extracts, proteins, purified protein preparation, or protein extracts prepared from induced cells of one or more bacteria, wherein the cell lysates, cell-free extracts, proteins, purified protein preparation, or protein extracts extract has monooxygenase activity (such as, for example, 1-hexene monooxygenase (1-HMO) or Styrene monooxynase activity including, but not limited to cell lysates, cell-free extracts, proteins, purified protein preparations, or protein extracts that comprise a monooxygenase including, but not limited to, 1-HMO) and delays a plant development process (including, but not limited to the ripening of climacteric fruit (including, but not limited to peach, pears, apples, avocados, peppers, tomatoes, grapes), protects ethylene sensitive species (including, but not limited to spinach, broccoli, and watermelons), and/or delays senescence of climacteric flowers (such as, for example, carnations).

Also disclosed herein are purified protein preparations, proteins, cell lysates, cell-free extracts, or protein extracts of any preceding aspect, wherein the one or more bacteria comprises Rhodococcus rhodochrous DAP 96253 strain, Rhodococcus rhodochrous DAP 96622 strain, Rhodococcus erythropolis, or combinations thereof.

In one aspect, disclosed herein are purified protein preparations, proteins, cell lysates, cell-free extracts, or protein extracts of any preceding aspect, wherein the purified protein, cell lysate, cell-free extract, or protein extract do not need an exogenously supplied cofactor (NADPH2 or FADH2) in order to be active, effective, retain, and/or sustain activity.

Also disclosed herein are purified protein preparations, proteins, cell lysates, cell-free extracts, or protein extracts of any preceding aspect, further comprising an epoxide hydrolase (OXase).

In one aspect, disclosed herein are purified protein preparations, proteins, cell lysates, cell-free extracts, or protein extracts of any preceding aspect, wherein the one or more bacteria are induced by exposure to an inducing agent selected from the group consisting of asparagine, glutamine, cobalt, urea, and mixtures thereof.

Also disclosed herein are containers comprising the purified protein preparation, protein, cell lysate, cell-free extract, or protein extract of any preceding aspect. In one aspect, the container can further comprise a fungicide, antibiotic, and/or post harvest aide.

In one aspect disclosed herein are methods for delaying a plant development process comprising exposing a plant or plant part to the purified protein preparation, protein, cell lysate, cell-free extract, or protein extract of any preceding aspect, and wherein the one or more bacteria are exposed to the plant or plant part in a quantity sufficient to delay the plant development process. For example, disclosed herein are methods for delaying a plant development process comprising exposing a plant or plant part to the purified protein, cell lysate, cell-free extract, or protein extract wherein the purified protein, cell lysate, cell-free extract, or protein extract comprises 1-hexene monooxygenase (1-HMO) activity and active against ethylene, and wherein the purified protein, cell lysate, cell-free extract, or protein extract are exposed to the plant or plant part in a quantity sufficient to delay the plant development process.

Also disclosed herein are methods for delaying a plant development process (such as, for example, fruit or vegetable ripening, flower senescence, or leaf abscission) of any preceding aspect, wherein the one or more bacteria are induced by exposure to an inducing agent selected from the group consisting of asparagine, glutamine, cobalt, urea, and mixtures thereof. In one aspect, disclosed herein are methods for delaying a plant development process of any preceding aspect, wherein the plant or plant part is directly or indirectly exposed to the purified protein preparation, protein, cell lysate, cell-free extract, or protein extract.

In one aspect, disclosed herein are methods for delaying a plant development process of any preceding aspect, wherein the plant part is a fruit (including, but not limited to climacteric fruit (such as, for example, peach, pears, apples, avocados, peppers, tomatoes, bananas, grapes, and bananas), nonclimacteric fruit, or vegetable). Also disclosed herein are methods for delaying a plant development process of any preceding aspect, wherein the flower is a carnation, rose, orchid, portulaca, malva, or begonia.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows examples of monooxygenase reactions

FIG. 2 shows an example of a flavin monooxygenase reaction.

FIG. 3 shows the degradation of NTA.

FIG. 4 shows EDTA degradation.

FIG. 5 shows illustrates the organization of proteins in a Monooxygenase cluster in P. oleovorans,

FIG. 6 shows a native PAGE of partially purified protein fractions using a Blue Dextran-DEAE cellulose anion exchange column, showing the monooxygenase and reductase subunits of 1-HMO. 1. Cell free lysate. 2. 180 mM NaCl Elution, 3.250 mM NaCl Elution.

FIG. 7 shows that bananas exposed to induced whole cells of R. rhodochrous DAP 96253 or to Isolated and purified 1-HMO enzymes for 13 days at ambient temperature. Panel A shows whole induced Cells 1× of R. rhodochrous, panel B shows control (100 μL ddi H2O); panel C shows 1×-1-HMO; and panel D shows ½× 1-HMO

FIG. 8 shows that bananas exposed to induced whole cells of R. rhodochrous DAP 96253 and to free isolated and purified 1-HMO and to immobilized 1-HMO formulations for 5 days at ambient temperature. Panel A shows Whole induced cells (1×). Panel B shows control (100 μL 50 mM phosphate buffer, pH 7). Panel C shows 500 μg 1-HMO Panel D shows 1-HMO immobilized in polyacrylamide matrix (PAM), Panel E shows 1-HMO immobilized in DEAE cellulose, Glutaraldehyde/Polyethylenimine

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes a single carrier as well as mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular purified protein preparation, protein, protein extract, cell lysate, and/or cell-free extract is disclosed and discussed and a number of modifications that can be made to a number of molecules including the purified protein preparation, protein, protein extract, cell lysate, and/or cell-free extract are discussed, specifically contemplated is each and every combination and permutation of purified protein preparation, protein, protein extract, cell lysate, and/or cell-free extract and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

The present invention provides methods for delaying a plant development process of interest (including delayed ripening in induced cells) comprising exposing a plant or plant part to a cell lysate, cell-free extract, purified protein preparation, protein, or protein extract from one or more bacteria. In particular embodiments, the methods are drawn to delaying a plant development process comprising exposing a plant or plant part to a cell lysate, cell-free extract, purified protein preparation, protein, or protein extract from one or more bacteria selected from the group consisting of Rhodococcus spp., Pseudomonas chloroaphis, Brevibacterium ketoglutamicum, and mixtures thereof, wherein the cell lysate, cell-free extract, protein, or protein extract are exposed to the plant or plant part in a quantity sufficient to delay the plant development process. Apparatuses for delaying a plant development process of interest and for practicing the methods described herein are further provided. The inventive methods and apparatuses of the invention may be used, for example, to delay fruit/vegetable ripening or flower senescence and to increase the shelf-life of fruit, vegetables, or flowers, thereby facilitating transportation, distribution, and marketing of such plant products.

It is disclosed and herein contemplated that one component of the cell lysate or cell-free extract that delays a plant development process (including, but not limited to the ripening of climacteric fruit, protects ethylene sensitive species, and/or delays senescence of climacteric flowers) are monooxygenases. Monooxygenase enzymes (MOs) comprise several broad classes of enzyme that, in general, catalyze the introduction of singlet oxygen from 02 into an organic compound with the second oxygen being incorporated into water. A number of major classes of MOs are recognized including: Cytochrome P450, Flavin Monooxygenases, Soluble double iron MOs (SDIMOs (e.g. soluble methane monooxygenase [sMMO], membrane bound copper (e.g. particulate MMO).

It must be noted that while many of these enzymes are inducible, and are involved in direct nutrition for the microorganisms that possess them, once induced these MOs can react with other compounds. (Methane bacteria can only grow on C1 compounds, MMO induced by these bacteria can also oxidize other C2-C4 compounds. This phenomenon in referred to Co-metabolism and is quite common.

A typical stylized reaction would include for example: the introduction of singlet oxygen into ethylene creating ethylene oxide.

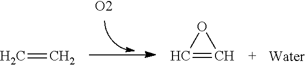

The genome of the bacterium Rhodococcus rhodochrous encodes for 19 different monooxygenases, with a total of 56 copies.

Induced cells of R. rhodochrous DAP 96253 also produce epoxide hydrolase(s) [OXase]. OXases exhibit broad substrate diversity (seen in animals, plants, bacteria, fungi). OXase can be viewed as typically adding water to an epoxide with the subsequent formation of a diol. Epoxides are extremely reactive and without prompt detoxification in animals and humans can cause serious damage and are implicated in carcinogenesis. OXase not only detoxify epoxide but bind to the epoxide(s) such that the epoxides can't exert their deleterious effects.

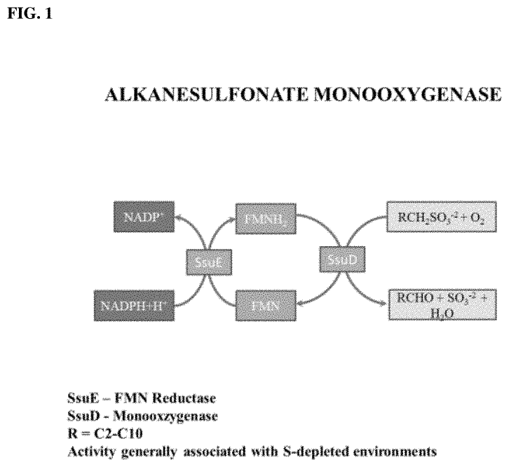

The reaction above illustrates the overall reaction but fails to convey the complexity of monooxygenase reactions. Examples of monooxygenase reactions are provided in FIG. 1.

The reaction FIG. 1 shows how a microorganism living in a sulfur depleted environment can obtain Sulfur from alkanesulfonates through the use of a monooxygenase. It is important to note that for the alkanesulfonate monooxygenase to function, hydrogen must be supplied from reduced pyridine nucleotide to flavin via a flavin mononucleotide reductase, which can then supply hydrogen to the monooxygenase. Without a supply of hydrogen, the reaction stops.

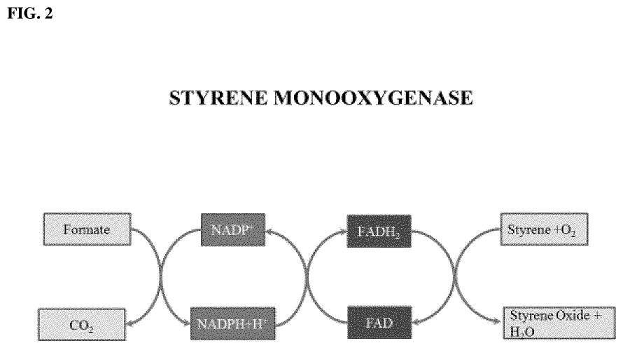

The reaction in FIG. 2 is an example of a Flavin Monooxygenase. In this type of monooxygenase reduced pyridine nucleotide (NADH2) is not involved.

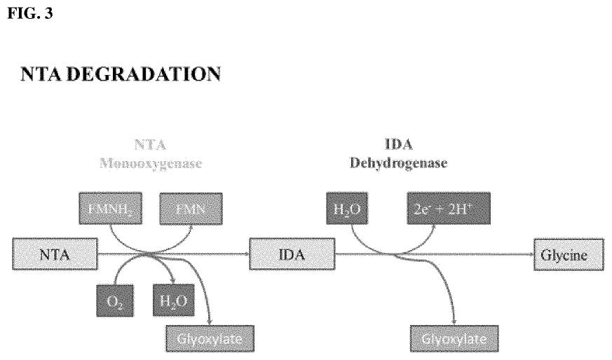

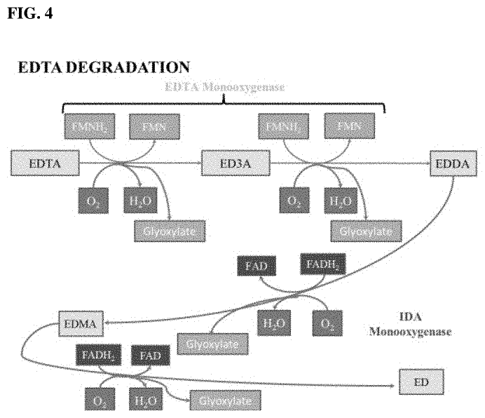

In the degradation of NTA (FIG. 3) a flavin monooxygenase is involved in only the first step. In EDTA degradation (FIG. 4) the same flavin monooxygenase catalyzes the first 2-steps in the degradation of EDTA. Typically, MMO catalyze only one-step.

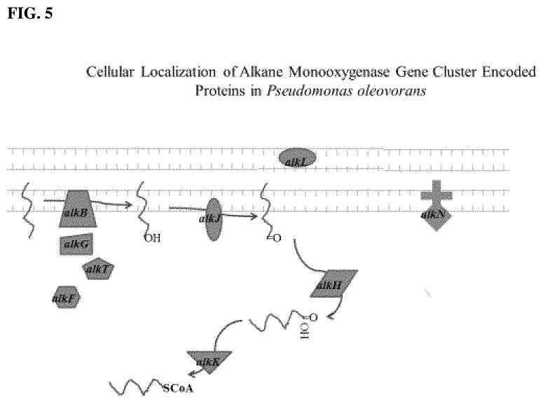

FIG. 5 illustrates the organization of proteins in a Monooxygenase cluster in P. oleovorans, which is typical for many monooxygenases, in that each monooxygenase has a unique reductase. Unlike Pseudomonas spp, in Rhodococcus spp, one reductase can serve multiple monooxygenases.

Thus, in one aspect, disclosed herein are purified protein preparations, proteins, protein extracts, cell lysates, and/or cell-free extracts comprising monooxygenase activity (such as, for example, 1-hexene monooxygenase (1-HMO) or Styrene monooxygenase activity).

Despite the extensive range of substrates attacked by monooxygenase, the uniqueness of the reactions and products, the use of commercial use of monooxygenases has been severely restricted because of the need to provide hydrogen (through reduced pyridine nucleotides or reduced flavin), and the increased cost and complexity of supplying exogenous form of reduced such as for example NAD, FMA, FAD.

In certain embodiments, the one or more bacteria are “induced” to exhibit a desired characteristic (e.g., the expression of a desired level of activity of an enzyme of the bacteria) by exposure or treatment with a suitable inducing agent. Inducing agents include, but are not limited to urea, methyl carbamate, cobalt, asparagine, glutamine, and combinations thereof. Optionally, the one or more bacteria are exposed to or treated with urea, methyl carbamate, methacrylamide, or acetamide. Optionally, the one or more bacteria are exposed to or treated with a mixture of inducing agents comprising urea or methyl carbamate and one or more of asparagine and cobalt. In some embodiments, the compositions and methods optionally exclude an inducing agent, such as cobalt.

The inducing agent, when used, can be added at any time during cultivation of the desired cells. For example, with respect to bacteria, the culture medium can be supplemented with an inducing agent prior to beginning cultivation of the bacteria. Alternately, the bacteria could be cultivated on a medium for a predetermined amount of time to grow the bacteria and the inducing agent could be added at one or more predetermined times to induce the desired enzymatic activity in the bacteria. Moreover, the inducing agent could be added to the growth medium (or to a separate mixture including the previously grown bacteria) to induce the desired activity in the bacteria after the growth of the bacteria is completed or during a second growth or maintenance phase.

While not intending to be limited to a particular mechanism, “inducing” the bacteria may result in the production or activation (or increased production or increased activity) of one or more of enzymes, such as nitrile hydratase, amidase, asparaginase, ACC deaminase, cyanoalanine synthase-like enzyme, styrene monooxygenase, 1-hexene monooxygenase (1-HMO), alkane monooxygenase, ammonium monooxygenase, methane monooxygenase, toluene dioxygenase, and/or cyanidase, and the induction of one or more of these enzymes may play a role in inhibiting or reducing fungal growth. “Nitrile hydratases,” “amidases,” “asparaginases,” “ACC deaminases” (including ACC deaminases in solution with ACC), “cyanoalanine synthase-like enzymes,” “AMO-type (alkane or ammonium) monooxygenases,” “methane monooxygenases,” “toluene dioxygenases,” and “cyanidases” comprise families of enzymes present in cells from various organisms, including but not limited to, bacteria, fungi, plants, and animals. Such enzymes are well known, and each class of enzyme possesses recognized enzymatic activities. In one aspect, disclosed herein are cell lysates, cell-free extracts, proteins, purified protein preparation, or protein extracts prepared from induced cells of one or more bacteria, wherein the cell lysates, cell-free extracts, proteins, purified protein preparation, or protein extracts extract has monooxygenase activity, including, but not limited to 1-hexene monooxygenase (1-HMO) activity and/or Styrene monooxygenase activity and delays a plant development process (including, but not limited to the ripening of climacteric fruit, protects ethylene sensitive species, and/or delays senescence of climacteric flowers). In some aspect, the cell lysates, cell-free extracts, proteins, purified protein preparation, or protein extracts comprise 1-HMO or Styrene MO.

The methods of inducing an enzymatic activity can be accomplished without the requirement of introducing hazardous nitriles, such as acrylonitrile, into the environment. Previously, it was believed that induction of specific enzyme activity in certain microorganisms required the addition of chemical inducers, for example, in the induction of nitrile hydratase activity in Rhodococcus rhodochrous and Pseudomonas chloroaphis, it was generally believed to be necessary to supplement with hazardous chemicals, such as acetonitrile, acrylonitrile, acrylamide, and the like. Using toxic hydrocarbons to induce enzymes such as for example NHase would likely make the fermentation products unacceptable for food and/or pharmaceutical use. However, enzymatic activity in nitrile hydratase producing microorganisms can be induced with the use of non-hazardous media additives, such as amide containing amino acids and derivates thereof, and optionally stabilized with trehalose. Optionally, asparagine, glutamine, or combinations thereof, can be used as inducers. Methods of inducing and stabilizing enzymatic activity in microorganisms are described in U.S. Pat. Nos. 7,531,343 and 7,531,344, which are incorporated herein by reference.

By growing R. rhodochrous DAP 96253 in a medium using carbohydrates, urea, and cobalt, induced cells were produced (Note no hydrocarbons, liquid, gaseous, or solid were included, contrary to accepted practice for inducing monooxygenases that are capable of oxidizing hydrocarbons.) that when place in proximity to climacteric fruit, were capable of delaying the ripening of that fruit. By comparing induced cells with non-induced cells, it was noted that a monooxygenase, specifically a monooxygenase with activity against 1-hexene was expressed to higher levels. It was further noted that when this 1-hexenemonooxygenase was isolated and purified from induced cells of R. rhodochrous DAP 96253, that the purified enzyme was capable of delaying the ripening of bananas. It was then shown that the purified 1-HMO can effectively delay the ripening of bananas without the need to exogenously supply either reduced pyridine nucleotide or reduced flavin. The purified 1-HMO has also been shown to contain low levels of an oxido-reductase. Oxido-reductases employ catalytic amounts of reduced pyridine nucleotide that are regenerated at the protein, obviating the need for an external supply of reduced pyridine. In some embodiments, the methods of induction and stabilization comprise culturing a nitrile hydratase producing microorganism in a medium comprising one or more amide containing amino acids or derivatives thereof, and, optionally, trehalose. Optionally, disclosed are methods for inducing nitrile-hydratase using a medium supplemented with amide containing amino acids or derivatives thereof, which preferably include asparagine, glutamine or a combination thereof. Optionally, disclosed are methods for inducing nitrile-hydratase using a nutritionally complete medium supplemented with only asparagine. Optionally, disclosed are methods for inducing nitrile-hydratase using a nutritionally complete medium supplemented with only glutamine Optionally, disclosed are methods for stabilizing nitrile-hydratase using a nutritionally complete medium supplemented with only trehalose. More particularly, the methods of induction and stabilization comprise culturing the microorganism in the medium and optionally collecting the cultured microorganisms or enzymes produced by the microorganisms. Thus, in one aspect, disclosed herein are purified protein preparations, proteins, cell lysates, cell-free extracts, or protein extracts of any preceding aspect, wherein the one or more bacteria are induced by exposure to an inducing agent selected from the group consisting of asparagine, glutamine, cobalt, urea, and mixtures thereof.

The 1-HMO complex isolated from induced cells of R. rhodochrous DAP 96253 represents the first time that a practical, active monooxygenase has been isolated where either exogenously supplied hydrogens (from NADPH2, FMNH2, or FADH2) or a separate protein needed to be added to the monooxygenase to make the monooxygenase functional for extended periods of time. In one aspect, disclosed herein are purified protein preparations, proteins, cell lysates, cell-free extracts, or protein extracts of any preceding aspect, wherein the purified protein, cell lysate, cell-free extract, or protein extract do not need an exogenously supplied cofactor (NADPH2 or FADH2) in order to be active or effective. Also disclosed herein are purified protein preparations, proteins, cell lysates, cell-free extracts, or protein extracts of any preceding aspect, further comprising an epoxide hydrolase (OXase).

The cell lysates, cell extracts, purified proteins preparations, proteins, and/or protein extracts disclosed herein as well as methods of their use for delaying a plant development process typically comprise exposing a plant or plant part to a cell lysate, cell-free extract, purified protein preparation, protein, or protein extract from one or more of the following bacteria: Rhodococcus spp., Pseudomonas chloroaphis, Brevibacterium ketoglutamicum, or a mixture containing any combination of these bacteria. In certain embodiments, the one or more bacteria include Rhodococcus spp., more particularly Rhodococcus rhodochrous DAP 96253 strain, Rhodococcus sp. DAP 96622 strain, Rhodococcus erythropolis, or mixtures thereof. As used herein, exposing a plant or plant part to a cell lysate, cell-free extract, purified protein preparation, protein, or protein extract from one or more bacteria includes, for example, exposure to intact bacterial cells bacterial cell lysates, bacterial extracts, purified protein preparation, protein, or protein extracts that possess enzymatic activity (i.e., “enzymatic extracts”), including, but not limited to monooxygenase activity (such as, for example, 1-HMO activity). Methods for preparing proteins, protein extracts, lysates and enzymatic extracts from cells, including bacterial cells, are routine in the art. The one or more bacteria used in the methods and apparatuses of the invention may at times be more generally referred to herein as the “catalyst.”

In accordance with the methods of the invention, the cell lysates, cell extracts, purified proteins preparations, proteins, and/or protein extracts are exposed to the plant or plant part in a quantity sufficient to delay the plant development process. “Exposing” a plant or plant part to a cell lysate, cell-free extract, purified protein preparation, protein, or protein extract from one or more bacteria of the invention includes any method for presenting a cell lysate, cell extract, purified proteins preparation, protein, and/or protein extract to the plant or plant part. Indirect methods of exposure include, for example, placing the cell lysate, cell extract, purified proteins preparation, protein, and/or protein extract or mixture thereof in the general proximity of the plant or plant part (i.e., indirect exposure). In other embodiments, the cell lysate, cell extract, purified proteins preparation, protein, and/or protein extract may be exposed to the plant or plant part via closer or direct contact. Furthermore, as defined herein, a “sufficient” quantity of the cell lysate, cell extract, purified proteins preparation, protein, and/or protein extract will depend on a variety of factors, including but not limited to, the particular cell lysate, cell extract, purified proteins preparation, protein, and/or protein extract utilized in the method, the form in which the cell lysate, cell extract, purified proteins preparation, protein, and/or protein extract is exposed to the plant or plant part, the means by which the cell lysate, cell extract, purified proteins preparation, protein, and/or protein extract is exposed to the plant or plant part, and the length of time of exposure. It would be a matter of routine experimentation for the skilled artisan to determine the “sufficient” quantity of the cell lysate, cell extract, purified proteins preparation, protein, and/or protein extract necessary to delay the plant development process of interest.

Although in particular embodiments the cell lysates, cell extracts, purified proteins preparation, proteins, and/or protein extracts are obtained from bacteria selected from the group consisting of Rhodococcus spp., Pseudomonas chloroaphis, Brevibacterium ketoglutamicum, any bacterium that delays a plant development process when exposed to a plant or plant part can be used in the present methods and apparatuses. For example, bacteria belonging to the genus Nocardia [see Japanese Patent Application No. 54-129190], Rhodococcus [see Japanese Patent Application No. 2-470], Rhizobium [see Japanese Patent Application No. 5-236977], Klebsiella [Japanese Patent Application No. 5-30982], Aeromonas [Japanese Patent Application No. 5-30983], Agrobacterium [Japanese Patent Application No. 8-154691], Bacillus [Japanese Patent Application No. 8-187092], Pseudonocardia [Japanese Patent Application No. 8-56684], Pseudomonas, and Mycobacterium are non-limiting examples of microorganisms that can be used according to the invention. Not all species within a given genus may exhibit the same properties. Thus, it is possible to have a genus generally known to include strains capable of exhibiting a desired activity (e.g., the ability to delay a particular plant development process such as, for example, fruit ripening) but have one or more species that do not generally exhibit the desired activity. In light of the disclosure provided herein and the general knowledge in the art, however, it would be a matter of routine experimentation for the skilled artisan to carry out an assay to determine whether a particular species possesses one or more of the desired activities.

Further, specific examples of bacteria useful for obtaining the cell lysates, cell extracts, purified protein preparations, proteins, and/or protein extracts disclosed herein include, but are not limited to, Nocardia sp., Rhodococcus sp., Rhodococcus rhodochrous, Klebsiella sp., Aeromonas sp., Citrobacter freundii, Agrobacterium rhizogenes, Agrobacterium tumefaciens, Xanthobacter flavas, Erwinia nigrifluens, Enterobacter sp., Streptomyces sp., Rhizobium sp., Rhizobium loti, Rhizobium legminosarum, Rhizobium merioti, Pantoea agglomerans, Klebsiella pneumoniae subsp. pneumoniae, Agrobacterium radiobacter, Bacillus smithii, Pseudonocardia thermophila, Pseudomonas chloroaphis, Pseudomonas erythropolis, Brevibacterium ketoglutamicum, Rhodococcus erythropolis, Nocardia farcinica, Pseudomonas aeruginosa. In particular embodiments, bacteria from the genus Rhodococcus, more specifically Rhodococcus rhodochrous DAP 96253 strain (ATCC Deposit No. 55899; deposited with the ATCC on Dec. 11, 1996), Rhodococcus sp. DAP 96622 strain (ATCC Deposit No. 55898; deposited with the ATCC on Dec. 11, 1996), Rhodococcus erythropolis, or mixtures thereof, are used in the methods and apparatuses of the invention. In one aspect, disclosed herein are purified protein preparations, proteins, cell lysates, cell-free extracts, or protein extracts of any preceding aspect, wherein the one or more bacteria comprises Rhodococcus rhodochrous DAP 96253 strain, Rhodococcus rhodochrous DAP 96622 strain, Rhodococcus erythropolis, or combinations thereof.

As used herein, “plant” or “plant part” is broadly defined to include intact plants and any part of a plant, including but not limited to fruit, vegetables, flowers, seeds, leaves, nuts, embryos, pollen, ovules, branches, kernels, ears, cobs, husks, stalks, roots, root tips, anthers, and the like. In particular embodiments, the plant part is a fruit, vegetable, or flower. In certain aspects of the invention, the plant part is a fruit, more particularly a climacteric fruit, as described in more detail below.

As shown herein, MO activity (for example 1-HMO activity) correlates with ethylene oxidation, and the disruption of ethylene biosynthesis (as in for example ripening). Thus, it is understood and herein contemplated that the purified protein preparations, proteins, cell lysates, cell-free extracts, or protein extracts disclosed herein can be used to delay plant development processes. Accordingly, disclosed the methods of the use disclosed purified protein preparations, proteins, cell lysates, cell-free extracts, or protein extracts are directed to delaying a plant development process, such as a plant development process generally associated with increased ethylene biosynthesis. “Plant development process” is intended to mean any growth or development process of a plant or plant part, including but not limited to fruit ripening, vegetable ripening, flower senescence, leaf abscission, seed germination, and the like. In particular embodiments, the plant development process of interest is fruit or vegetable ripening, flower senescence, or leaf abscission, more particularly fruit or vegetable ripening. As defined herein, “delaying a plant development process,” and grammatical variants thereof, refers to any slowing, interruption, suppression, or inhibition of the plant development process of interest or the phenotypic or genotypic changes to the plant or plant part typically associated with the specific plant development process. For example, when the plant development process of interest is fruit ripening, a delay in fruit ripening may include inhibition of the changes generally associated with the ripening process (e.g., color change, softening of pericarp (i.e., ovary wall), increases in sugar content, changes in flavor, general degradation/deterioration of the fruit, and eventual decreases in the desirability of the fruit to consumers, as described above). One of skill in the art will appreciate that the length of time required for fruit ripening to occur will vary depending on, for example, the type of fruit and the specific storage conditions utilized (e.g., temperature, humidity, air flow, etc.). Accordingly, “delaying fruit ripening” may constitute a delay of 1 to 90 days, particularly 1 to 30 days, more particularly 5 to 30 days, most specifically 5-14 days Methods for assessing a delay in a plant development process such as fruit ripening, vegetable ripening, flower senescence, and leaf abscission are well within the routine capabilities of those of ordinary skill in the art and may be based on, for example, comparison to plant development processes in untreated plants or plant parts. In certain aspects of the invention, delays in a plant development process resulting from the practice of the present methods may be assessed relative to untreated plants or plant parts or to plants or plant parts that have been treated with one or more agents known to retard the plant development process of interest. For example, a delay in fruit ripening resulting from performance of a method of the invention may be compared to fruit ripening times of untreated fruit or fruit that has been treated with an anti-ripening agent, such as those described herein above.

Accordingly, in one aspect disclosed herein are methods for delaying a plant development process (including but not limited to the disruption of ethylene biosynthesis; delayed fruit or vegetable ripening, flower senescence, or leaf abscission; and/or protects ethylene sensitive species) comprising exposing a plant or plant part to the purified protein preparation, protein, cell lysate, cell-free extract, or protein extract of any preceding aspect, and wherein the one or more bacteria are exposed to the plant or plant part in a quantity sufficient to delay the plant development process. For example, disclosed herein are methods for delaying a plant development process comprising exposing a plant or plant part to the purified protein, cell lysate, cell-free extract, or protein extract wherein the purified protein, cell lysate, cell-free extract, or protein extract comprises 1-hexene monooxygenase (1-HMO) activity, and wherein the purified protein, cell lysate, cell-free extract, or protein extract are exposed to the plant or plant part in a quantity sufficient to delay the plant development process.

Also disclosed herein are methods for delaying a plant development process (including but not limited to the disruption of ethylene biosynthesis; delayed fruit or vegetable ripening, flower senescence, or leaf abscission; and/or protects ethylene sensitive species) of any preceding aspect, wherein the one or more bacteria are induced by exposure to an inducing agent selected from the group consisting of asparagine, glutamine, cobalt, urea, and mixtures thereof. In one aspect, disclosed herein are methods for delaying a plant development process of any preceding aspect, wherein the plant or plant part is directly or indirectly exposed to the purified protein preparation, protein, cell lysate, cell-free extract, or protein extract.

In one aspect, disclosed herein are methods for delaying a plant development process of any preceding aspect, wherein the plant part is a fruit (including, but not limited to climacteric fruit (such as, for example, peach, pears, apples, avocados, peppers, tomatoes, bananas, grapes, and bananas), nonclimacteric fruit, or vegetable). Also disclosed herein are methods for delaying a plant development process of any preceding aspect, wherein the flower is a carnation, rose, orchid, portulaca, malva, or begonia. It is understood and herein contemplated that one mechanism for contacting the disclosed cell lysates, cell extracts, proteins, protein extracts, and purified protein preparations is through the use of containers that have said cell lysates, cell extracts, proteins, protein extracts, and purified protein preparations embedded in them or coated on them. Thus, in one aspect, disclosed herein are containers comprising the purified protein preparation, protein, cell lysate, cell-free extract, or protein extract of any preceding aspect. In one aspect, the container can further comprise a fungicide, antibiotic, and/or post harvest aide.

1. Homology/Identity

It is understood that one way to define any known variants and derivatives or those that might arise, of the disclosed genes and proteins herein is through defining the variants and derivatives in terms of homology to specific known sequences. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms.

Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

2. Peptides

a) Protein Variants

As discussed herein there are numerous variants of the 1-hexene monooxygenase (1-HMO) or Styrene monooxygenase (MO) protein that are known and herein contemplated. In addition, to the known functional strain variants there are derivatives of the 1-HMO and Styrene MO proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 2
Amino Acid Substitutions
Original Residue Exemplary Conservative Substitutions,
others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl. Additionally, nitrile hydratase (NHase) shows post translational modifications in the cysteine residues. For two of the three cysteines, the sulfur is oxidized. The third residue remains unchanged.

It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein from which that protein arises is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way.

Molecules can be produced that resemble peptides, but which are not connected via a natural peptide linkage. For example, linkages for amino acids or amino acid analogs can include CH₂NH—, —CH₂S—, —CH₂—CH₂—CH═CH—(cis and trans), —COCH₂—CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CHH₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

### C. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Delayed Ripening of Climacteric Fruit, Post-Harvest by a Monooxygenase Exhibiting 1-Hexene Monooxygenase Activityisolated from Induced Cells of Rhodococcus rhodochrous DAP 96253

Post-harvest losses of fruit and vegetables in the U.S. are considerable. The USDA estimates that on average, post-harvest losses of fruits and vegetables are 40%, which translates to over 11 billion pounds of food waste annually or 275 lb. for every person in the US. It is estimated that the ability to reduce these losses by just 15% can provide sufficient fresh fruit and vegetables to feed 25 million people. At the same time, the demand for fresh produce (versus processed) continues to increase, with the consumer demand for organic fresh produce increasing even faster. By addressing the current level of post-harvest losses in fresh fruit and vegetables it is possible to address the increasing demand for fresh produce without increasing current farm-land acreage, fertilizer usage or energy costs.

Induced cells of Rhodococcus rhodochrous DAP 96253 were capable of extending the shelf-life of a number of climacteric fruit (e.g. peaches and bananas). The shelf life of tomatoes, grapes, strawberries and raspberries has similarly been extended. These experiments clearly showed that the induced cells of R. rhodochrous DAP 96253 did not need to be in direct contact with the produce to delay the ripening of that produce.

Ripening (and injury response) in climacteric fruits and vegetables is initiated by the biosynthesis of ethylene, HCN and CO₂, known as the Yang Cycle and for many years efforts to control ripening have been directed towards either limiting ethylene synthesis and/or ethylene recognition. It is well known that ethylene biodegradation can be catalyzed through the action of many different monooxygenases, and not just alkane monooxygenases. It is equally well known that monooxygenases implicated in the degradation of ethylene require an exogenously supplied reduced cofactor, such as for example, NAD, FAD, or Rubredoxin. While the regeneration of the required, reduced cofactor would not represent a problem for actively growing cells, this is a serious problem for commercial processes using the isolated enzyme or for dead and/or resting cells. Induced cells of R. rhodochrous killed during immobilization with glutaraldehyde/polyethylene imine, were still capable of delayed ripening of climacteric fruit. This argues against the involvement of a conventional monooxygenase/alkene monooxygenase in the delayed ripening seen with induced dead cells of R. rhodochrous DAP 96253.

In this report, is described how several enzymes were identified as being present when comparing induced and non induced cells, and then isolating and purifying these enzymes. understand delayed ripening led to the discovery of an unique monooxygenase possessing 1-HMO activity. This approach led to the discovery of hexene monooxygenase (1-HMO) activity from induced cells of R. rhodochrous DAP 96253, which was capable of delaying the ripening of bananas, and which did not require/need exogenously supplied pyridine nucleotide for delaying the ripening of bananas.

a) Approach/Materials and Methods.

(1) Microorganism.

R. rhodochrous DAP 96253 has been deposited with the ATCC, Manassas Va. under the terms of Budapest Treaty and has been assigned the accession number ATCC #55899, and is the subject of several issued patents. Cultures are maintained on YEMEA, and are induced according to U.S. Pat. Nos. 7,531,344; 7,943,549; and 8,389,441 which are incorporated herein by reference for their teachings of maintaining cultures on YEMEA and induction.

(2) Fermentation.

For consistency in preparing sufficient cell mass to isolate and purify monooxygenase of interest, induced cells of R. rhodochrous DAP 96253 were prepared using a fed-batch process in 20 and 30-liter Sartorius BioStat C+ bioreactor (SCADA software MFCS-win). Fermentation runs were conducted using a 2-sage process: where Stage 1 is run the batch mode until substrate depletion is observed, followed by the 2^{nd} stage which was run in the fed-batch mode with nutrient and inducer addition accomplished incrementally over time. In stage 1 (batch mode) dilute YEMEA broth or R3A (BBL) were used as the medium. In Stage-2 (Fed-batch Mode) glucose was added incrementally to achieve concentrations typical of that seen in YEMEA broth. Urea (7.5-16 g/1) and Cobalt, as CoC126H₂O, (50 mg/1) also were added. All media formulations were animal free and in the media formulations employed in this work, all animal protein was replaced on an equivalent basis using cottonseed protein (Friesland North America). Air was constant at 5 or 7.5 LPM. O₂ was sparged on demand, in conjunction with agitation to maintain dissolved Oxygen (%) above K_{CRIS}. pH was maintained at 7.0 (0.1 deadband) by the automatic addition of 1N hydrochloric acid or 2N sodium hydroxide or Sulfuric Acid. Induced cells were harvested and decanted with the aid of a Carr Pilot PowerFuge (Pneumatic Scale Angelus, Stow, Ohio). Cells not immediately used were stored frozen (−20° C.) in 150-gram aliquots as decanted cell paste

(3) Chemicals

Unless otherwise stated all chemicals were obtained from Sigma/Aldrich.

(4) Enzyme Activity Determination.

(a) Nitrile Hydratase/Amidase

Nitrile hydratase was determined by first converting the nitrile substrate (acrylonitrile) to the corresponding amide and then adding amidase (Sigma) to convert the amide to the corresponding acid, with the concomitant release of ammonia.

(b) 1-Hexene Monooxygenase Activity

99. 1-Hexene specific monooxygenase activity was determined as follows. Suspended cells of R. rhodochrous DAP 96253, 20 to 100 mg (cell dry weight) in 5 ml of phosphate buffer (pH 7.0) were added to a 40 ml dark amber glass vial. 1-hexene (200 ul) was added to the amber vial. 4-(4-nitrobenzyl) pyridine [NBP] 500 ul (from a 100 mM solution in ethylene glycol) was then added to a 4 ml transparent glass vial, which was then placed inside of the amber vial. The outer vial was then crimped sealed with a Teflon-faced butyl rubber stopper. The vials were then incubated at 30 C, with shaking (150 rpm—in a NBS G76 gyratory shaker) for 24 hours after which 500 μl of triethylamine solution (1:1 in acetone) was added to the inner vial, and the absorbance was measured at 600 nm [BioPhotomer plus (Eppendorf, Hauppauge, N.Y.)]. Standards of 1,2 epoxyhexane (1-5 mM), in 50 mM phosphate buffer, pH 7.0, were made from a stock solution of 10 mM 1,2-epoxyhexane solution (3.01 ul 1,2-epoxyhexane into 2.5 mL of acetone).

(5) Immobilization of Cells

The calcium alginate-cell immobilization method described in Methods in Enzymology was employed to prepare beads as follows. Induced cells of R. rhodochrous DAP 96253 grown on YEMEA supplemented with glucose and urea or glucose urea and cobalt according to the methods in Pierce et al, were suspended in 50 mM Tris buffer (pH 7). Equal volumes of cell suspension were mixed with an equal volume of sodium alginate/polyvinyl alcohol to achieve a final concentration 1.25% each of alginate and PVA. The solution was then introduced dropwise into a 0.1M Calcium solution and allowed to sit for 30 minutes, at which time the beads were recovered and washed in 50 mM Tris buffer (pH 7.0).

(6) Analysis of R. rhodochrous DAP 96253 Genomic-DNA

R. rhodochrous DAP 96253 was grown under inducing and non-inducing conditions. DNA was extracted using the TRIzol® extraction kit according to the manufacturer's instructions (Thermofisher); the DNA was quantified using a NanoDrop™ microvolume spectrophotometer (Thermofisher), and then sent to the Forest Products Laboratory for library preparation and sequencing. Raw sequence data returned by the Forest Products Laboratory was then subjected to DE NOVO assembly using SPAdes. Annotation was performed using RAST (Rapid Annotation using Subsystem Technology, ver. 2) and alignments were performed using CLC genomics workbench, 11 (QIAGEN).

(7) Purification of 1-HMO

Induced cells (150 g cell packed wet weight) of Rhodococcus rhodochrous 96253 were washed 1× using wash buffer (MOPS 25 mM, pH 7.5) and then resuspended using Buffer A (MOPS 25 mM, Glycerol 5%, DTT 5 mM, pH 7.5) in the ratio 1:2. The suspended cells were homogenized at 4 C and 700 bar using an APV Homogenizer (SPX Flow Technology, Charlotte, N.C.). After 4 passes the extract was centrifuged at 10,000 RPM at 4 C for 30 minutes in a Beckman Avanti J-20 XPI centrifuge (Beckman Coulter Life Sciences, Indianapolis, Ind.). The supernatant was collected and subjected to dialysis at 4 C for 16 hours against Equilibration Buffer (25 mM MOPS, 15% glycerol, 5 mM DTT, 1% MgSO₄.7H₂O, pH 7.5).

The resultant dialysate was then purified using a GE ÄKTA Explorer™ (GE Biosciences, Piscataway, N.J.) using a Blue Dextran DEAE column (Diethyl Aminoethyl Cellulose column) for final 1-HMO purification. 25 ml of the dialyzed supernatant was taken and subjected to Blue Dextran DEAE cellulose column chromatography. The column was equilibrated using 150 mM Phosphate buffer, pH 7.5. The enzyme was then eluted using linear gradient of 0.25M, 0.5M and 1M NaCl, all at pH 7.5 in 150 mM phosphate buffer. The GE ÄKTA Explorer™ was controlled by UNICORN® 5.11 platform. Protein peaks were determined by UV280 and UV260, and both wavelengths were detected by a UV-900 detector from the system. Samples were collected for BCA total protein analysis, Gel Electrophoresis, and for the specific enzyme assays.

(8) Blue Dextran DEAE Cellulose Column.

A 5 ml HiTrap Fast Flow DEAE (Diethyl Aminoethyl Cellulose (GE Healthcare Life Sciences) column was injected with 5 ml of Blue dextran and allowed to sit at 4° C. for 24 hours. The above DEAE column is a weak anion exchanger that has positively charged resin and binds to negatively charged protein. Blue Dextran minimizes the void volume, thus exposing only the needed column spaces, and thus maximizing the binding capacity of proteins. After 24 hours, the Blue-Dextran DEAE column was taken and equilibrated using above mentioned 150 mM phosphate buffer.

(9) Cell Preparations for Testing Delayed Ripening

Unless otherwise stated when whole cell preparations were tested for delayed ripening activity, the cell preparations were placed in sterile M9 minimal salts medium: salts: 6 g of Na₂HPO₄, 6 g; KH₂PO₄, 3 g; NaCl, 0.5 g and 1.0 g of NH₄Cl, 1.0 g are dissolved into 980 ml of DI water and sterilized. To this sterile solution, 10 ml of 0.1M MgSO₄ and 10 ml of 0.01M CaCl₂ were added. The cells in buffer were then placed in an open dish (Petrie dish, 100 mm) in proximity to the fruit to be tested. Equivalent amounts of cells in Alginate/PVA beads were placed in the same type of open dishes, suspended with Tris-HCL buffer and then placed in proximity to fruit. (At no time were the cells or immobilized cells in contact with the fruit.)

(10) Delayed Ripening

Selected fruit (e.g. bananas) were obtained in the firm ripe state. Three hands of bananas (at least 6 fingers per hand) were separated so that two bananas from each hand were placed in control, standard and experimental plastic trays, resulting in six bananas in each tray. (This was done to normalize differences seen from hand to hand. Bananas were then maintained at room temperature throughout the test. Purified enzyme with or without cofactor was then placed into a small uncovered Petri dish (35 mm) into the tray with the bananas but not in direct contact with the bananas.

b) Results
(1) Investigation of Proteins Showing Increased Abundance when Induced.

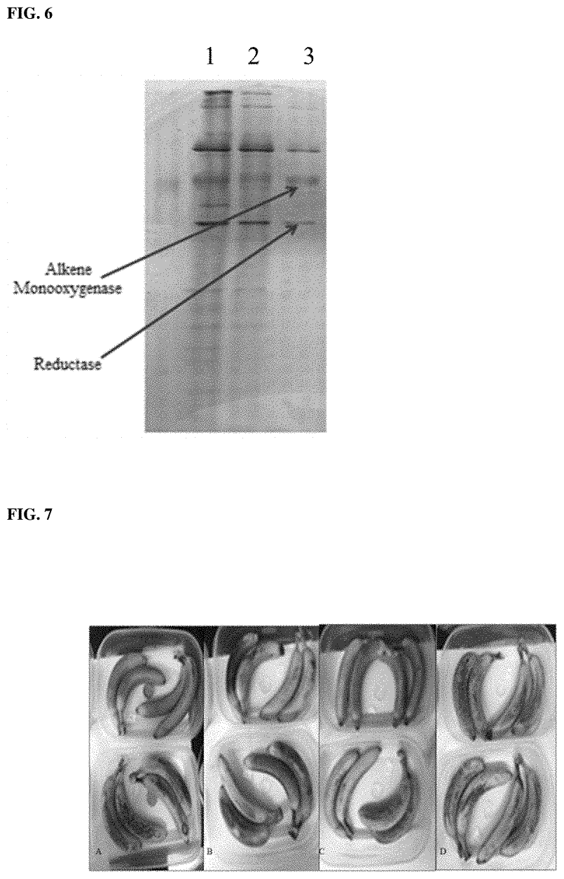

In parallel, experiments were conducted in which the proteins from induced cells of R. rhodochrous DAP 96253 were compared to non-induced cells. Where increased levels of protein expression were observed the protein from the induced cells was isolated, sequenced, and separately purified. When analyzing induced cells using a variety of potential monooxygenase substrates, there was a strong correlation between cells with delayed ripening activity and the ability to oxidize 1-hexene. By using native gels, and assaying the enzymatic activities of those proteins present in increased abundance, several additional monooxygenases were identified: 1) a protein with styrene monooxygenase activity and 2) a protein with 1-hexene monooxygenase (1-HMO) activity. Additionally, a monooxygenase having 1-HMO activity was isolated and purified. The native gel for the monooxygenase and a reductase are shown in the gel image in FIG. 6. rom native gels, the individual bands were excised, digested and the fragments analyzed by MALDI-TOF with both the 1-hexene monooxygenase and the reductase confirmed. Enzyme assays confirmed the respective identities.

(2) Delayed Ripening by Purified 1-HMO

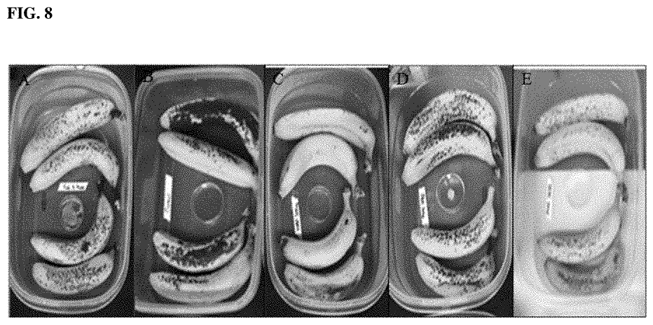

Purified 1-HMO, in buffer, with and without either NADH or FADH was tested to determine if the purified enzyme alone could delay the ripening of bananas. FIGS. 7 and 8 below clearly show that purified 1-HMO was capable of delaying the ripening of bananas, and that exogenously supplied NADH or FADH were not required for this activity. (Separate assays, conducted in parallel using 1-hexene as the substrate also showed that exogenously supplied NADH or FADH were not required for oxidation of 1-hexene to the corresponding epoxide.)

Also shown in FIG. 8, delayed ripening experiments were conducted using entrapped/immobilized—purified 1-HMO. Results are shown for 1-HMO entrapped in polyacrylamide (PAM) and 1-HMO passively attached to DEAE-cellulose and then immobilized using glutaraldehyde-polyethylenimine Both immobilized formulations of purified 1-HMO show delayed ripening of bananas.

c) Discussion

While the isolation and subsequent sequence analysis of the genomic-DNA from R. rhodochrous DAP 96253 (plasmid-DNA was not isolated and sequenced) showed the presence of a great number and diversity of monooxygenases, physiological and biochemical testing of induced cells of R. rhodochrous DAP 96253 showed only a few monooxygenases induced. Specifically, it was noted that robust delayed ripening activity of induced cells was correlated with elevated levels of 1-hexene monooxygenase activity (1-HMO). In parallel experiments, SDS-PAGE electrophoresis of total soluble protein obtained from induced and non-induced cells of R. rhodochrous DAP 96253 also showed only a few proteins with increased abundance. Extraction of these candidate proteins, and their subsequent preparation, digestion, and partial sequence determination by MALDI-TOF-TOF mass spectroscopy resulted in the identification of several MOs of interest. The purification of these MOs of interest, and their subsequent sequence determination, and enzymatic testing revealed the identification of a single unique protein possessing both 1-HMO activity, and which did not require an exogenously supplied cofactor. When purified the protein exhibiting 1-HMO activity was presented in either 150 mM phosphate buffer (pH 7.0) alone or when entrapped/immobilized in calcium alginate-PVA, PAM, or DEAE cellulose/glutaraldehyde/PEI was placed in proximity to bananas, delayed ripening was observed in all cases. It was noted that neither the delayed ripening activity nor activity against 1-hexene was required exogenously supplied reduced NAD or FAD. The presence or absence of NAD or FAD had no effect on the ability of the isolated enzyme to delay fruit ripening activity or on the ability to oxidize 1-hexene. This is the first report of a monooxygenase possessing activity against ethylene and/or 1-hexene that does not require exogenously supplied reduced pyridine nucleotide for activity of the purified enzyme or for activity in dead cells. While other MOs present in live, growing cells of R. rhodochrous DAP 96253 may be involved in the delayed ripening of climacteric fruit, the enzyme, exhibiting 1-HMO activity that was isolated from induced cells of R. rhodochrous DAP 96253 clearly shows the capability to delay ripening in dead cells, and when the isolated enzyme is used alone. Research is currently underway to compare the 1-HMO enzyme isolated from induced cells of R. rhodochrous DAP 96253 to other MOs present in strain 96253 and other MOs in other Rhodococcus strains and related mycolata. The relationship between the 1-HMO enzyme and the high-mass, cobalt containing nitrile hydratase and amidase which are significantly expressed in induced cells of R. rhodochrous DAP 96253 also is being investigated.

### D. References

Belshazzar S (2017) Process improvement for purification and characterization of select enzymes from distinct strains of Rhodococcus rhodochrous DAP 96453 and 96622. Dissertation. Georgia State University
Bucke C (1987) Cell immobilization in calcium alginate. In: Methods in Enzymology, Vol 135: Immobilized Enzymes and Cells, Part B. Colowick S P, Kaplan N O (ed) Academic Press, NY, pp 175-194
Bunescu A, Besse-Hoggan P, Sancelme, M, Mailhot G, Delort A-M (2008) Fate of the Nitrilotriacetic Acid-Fe (III) Complex during Photodegradation and Biodegradation by Rhodococcus rhodochrous. Appl Environ Microbiol 74: 6320-6326
Buzby J C, Wells H F, Hyman J, (2014) The Estimated Amount, Value, and Calories of Postharvest Food Losses at the Retail and Consumer Levels in the United States.” USDA Economic Research Service, Washington, D.C., USA
Chiciudean I, Nie Y, Tanase N M, Stoica I, X-L. Wu X L (2018) Complete genome sequence of Tsukamurella sp. MH1: a wide-chain length alkane-degrading actinomycete. J Biotechnol 268: 1-5
Chien C-C, Leadbetter E R, Godchaux W (1999) Rhodococcus spp. utilize taurine (2-aminoethanesulfonate) as sole source of carbon, energy, nitrogen and sulfur for aerobic respiratory growth. FEMS Microbiol Lett 176:333-337
Dietz A, Thayer D W (1980) in: Dietz A, Thayer D W (eds.) Actinomycete taxonomy SIM Special Publication No. 6. Society for Industrial Microbiology, Arlington, Va. pp 227-291
Erdlenbruch B N S, Kelly D P, Murrell J C (2001) Alkanesulfonate degradation by novel strains of Achrombacter xylosoxidans, Tsukamurella wratislaviensis and Rhodococcus sp., and evidence for an ethanesulfonate monooxygenase in A. xylosoxidans strain AE4. Arch Microbiol 176: 406-414
Fawcett J K, Scott J E, (1960) A rapid and precise method for the determination of urea. J Clin Path 13: 156-159
Golding J B, Shearer D, Wyles S G, McGlasson W B (1998) Application of 1-MCP and propylene to identify ethylene-dependent ripening processes in mature banana fruit. Postharvest Biol 14: 87-98
Goldstein L (1987) Polymers bearing isonitrile functional groups as supports for enzyme immobilization. In: Methods in Enzymology, Vol 135—Immobilized Enzymes and Cells, Part B. Colowick S P, Kaplan N O (ed) Academic Press, NY, pp 90-102
Jiang W (2016) The preliminary study on the role of 1-hexene specific alkene monooxygenase in delayed fruit ripening by Rhodococcus rhodochrous DAP 96253. Dissertation. Georgia State University
Kumar R, Verma H, Haider S, Bajaj A et al (2017) Comparative Genomic Analysis Reveals Habitat-Specific Genes and Regulatory Hubs within the Genus Novosphingobium. MSystems. DOI: 10.1128/mSystems.00020-17
McClay K, Fox B G, Steffan R J (2000) Toluene monooxygenase catalyzed epoxidation of alkenes. Appl Environ Microbiol 66: 1877-1882
Nilsson K, Mosbach K (1987) Tresyl, Chloride-activated supports for enzyme immobilization. In: Methods in Enzymology, Vol 135: Immobilized Enzymes and Cells, Part B. Colowick S P, Kaplan N O (ed) Academic Press, NY, pp 65-78
Piccolo L L, DePasquale C, Fodale R, Puglia A M, Quatrini P (2011) Involvement of an alkane hydroxylase system of Gordonia sp. Strain SoCg in degradation of solid n-alkanes. Appl Environ Microbiol 77: 1204-1213
Pierce G E, Drago G K, Ganguly S (2009) Induction and stabilization of enzymatic activity in microorganisms. U.S. Pat. No. 7,531,343
Pierce G E, Drago G K, Ganguly S (2009) Induction and stabilization of enzymatic activity in microorganisms. U.S. Pat. No. 7,531,344
Pierce G E, Drago G K, Ganguly S (2011) Biological-based catalyst to delay plant development processes. U.S. Pat. No. 7,943,549
Pierce G E, Drago G K, Ganguly S (2013) Biological-based catalyst to delay plant development processes. U.S. Pat. No. 8,389,441
Pierce G E, Tucker T M, Wang C, Swensen K, Crow Jr S A (2014) Delayed ripening of climacteric fruit by catalysts prepared from induced cells of Rhodococcus rhodochrous DAP 96253—A case for the biological modlulation of Yang-cyle driven processes by a Prokaryote. Ind Biotechnol 10: 354-362
Pierce, G E, Drago G K, Ganguly, Tucker T M, Hooker J W, Jones S, Crow Jr S A (2011) Preliminary report on a catalyst derived from induced cells of Rhodococcus rhodochrous strain DAP 96253 that delays the ripening of selected climacteric fruit: bananas, avocados, and peaches. J Ind Microbiol Biotechnol 38:1567-1573
Salamanca D, Karande R, Schmid A, Dobslaw D (2015) Novel cyclohexane monooxygenase from Acidovorax sp. CHX100. Appl Microbiol Biotechnol 99: 6889-6897
Sisler E, Serek C M, Dupille E (1996) Comparison of cyclopropane, 1-methylcyclopropene, and 3,3-dimethylcyclopropene as ethylene antagonists in plants. Plant Growth Regul 18: 169-174
Small F J, Ensign S A (1995) Carbon dioxide fixation in the metabolism of propylene and propylene oxide by Xanthobacter strain Py2. J Bacteriol 177: 6170-6175
Small, F J, Ensign S A (1997) Alkene Monooxygenase from Xanthobacter Strain Py2. The J. of Biol. Chem. 272: 24913-24920
Srivastava (2001) Plant Growth and Development: Hormones and Environment (Academic Press, New York)
USDA, ERS. 2017, Organic Market Overview. (Apr. 4, 2017).
Whyte L G, Smits T H M, Labbbe D, Witholt B, Greer C W, van Beilen J B (2002) Gene cloning and characterization of multiple alkane hydroxylase systems in Rhodococcus strains Q15 and NRRL B-16531. Appl Environ Microbiol 68: 5933-5942
Yang S F, Hoffman N E (1984) Ethylene biosynthesis and its regulation in higher plants. Ann Rev Plant Physiol 35: 155-189

What is claimed is:

1. A purified protein, cell lysate, cell-free extract, or protein extract prepared from induced cells of one or more bacteria, wherein the cell lysate or cell-free extract has 1-hexene monooxygenase (1-HMO) activity and delays the ripening of climacteric fruit, protects ethylene sensitive species, and/or delays senescence of climacteric flower.

2. The purified protein, cell lysate, cell-free extract, or protein extract of claim 1, wherein the cell lysate or cell-free extract has 1-hexene monooxygenase (1-HMO) activity and protects ethylene sensitive species.

3. The purified protein, cell lysate, cell-free extract, or protein extract of claim 1, wherein the cell lysate or cell-free extract has 1-hexene monooxygenase (1-HMO) activity and delays senescence of climacteric flower.

4. The purified protein, cell lysate, cell-free extract, or protein extract of claim 1, wherein the one or more bacteria comprises Rhodococcus rhodochrous DAP 96253 strain, Rhodococcus rhodochrous DAP 96622 strain, Rhodococcus erythropolis, or combinations thereof.

5. The purified protein, cell lysate, cell-free extract, or protein extract of claim 1, wherein the purified protein, cell lysate, cell-free extract, or protein extract do not need an exogenously supplied cofactor (NADPH2 or FADH2) in order to be active or effective.

6. The purified protein or protein extract of claim 1 wherein the protein or protein extract comprises 1-hexene monooxygenase (1-HMO) or styrene monooxygenase.

7. The purified protein, cell lysate, cell-free extract, or protein extract of claim 1, further comprising an epoxide hydrolase (OXase).

8. The purified protein, cell lysate, cell-free extract, or protein extract of claim 1, wherein the one or more bacteria are induced by exposure to an inducing agent selected from the group consisting of asparagine, glutamine, cobalt, urea, and mixtures thereof.

9. The purified protein, cell lysate, cell-free extract, or protein extract of claim 8, wherein the one or more bacteria are induced by exposure to asparagine, cobalt, and/or urea.

10. A container comprising the purified protein, celllysate, cell-free extract, or protein extract of claim 1.

11. The container of claim 10, further comprising a fungicide, antibiotic, and/or post harvest aide.

12. A method for delaying a plant development process comprising exposing a plant or plant part to the purified protein, cell lysate, cell-free extract, or protein extract of claim 1, and wherein the one or more bacteria are exposed to the plant or plant part in a quantity sufficient to delay the plant development process.

13. A method for delaying a plant development process comprising exposing a plant or plant part to the purified protein, cell lysate, cell-free extract, or protein extract wherein the purified protein, cell lysate, cell-free extract, or protein extract comprises 1-hexene monooxygenase (1-HMO) activity, and wherein the purified protein, cell lysate, cell-free extract, or protein extract are exposed to the plant or plant part in a quantity sufficient to delay the plant development process.

14. The method of claim 13, wherein the one or more bacteria are induced by exposure to an inducing agent selected from the group consisting of asparagine, glutamine, cobalt, urea, and mixtures thereof.

15. The method of claim 14, wherein the one or more bacteria are induced by exposure to asparagine, cobalt, and/or urea.

16. The method of claim 13, wherein the plant or plant part is indirectly exposed to the purified protein, cell lysate, cell-free extract, or protein extract.

17. The method of claim 13, wherein the plant or plant part is directly exposed to the purified protein, cell lysate, cell-free extract, or protein extract.

18. The method of claim 13, wherein the plant development process is fruit or vegetable ripening.

19. The method of claim 18, wherein the fruit is a climacteric fruit selected from the group consisting of bananas, peaches, and avocados.

20. The method of claim 13, wherein the plant part is a flower, and the plant development process is flower senescence or leaf abscission.

21. The method of claim 20, wherein the flower is a carnation, rose, orchid, portulaca, malva, or begonia.
